(19) United States
(12) Patent Application Publication
(10) Pub. No.: US 2013/0095521 A1
(43) Pub. Date: Apr. 18, 2013

(54) METHOD FOR TESTING AND MONITORING THE STERILITY OF PLANT PRODUCTION UNITS

(71) Applicant: Lidia de Rigo, Padova (IT)

(72) Inventor: Lidia de Rigo, Padova (IT)

(73) Assignee: Fresenius Kabi Anti-Infectives S.r.l., Milano (IT)

(21) Appl. No.: 13/650,632

(22) Filed: Oct. 12, 2012

Related U.S. Application Data
(63) Continuation of application No. 13/473,016, filed on May 16, 2012, now abandoned.

(30) Foreign Application Priority Data
May 17, 2011 (IT) MI2011A000864

(51) Int. Cl. C12Q 1/22

(52) U.S. Cl. CPC C12Q 1/22
USPC 435/36; 435/34

(57) ABSTRACT

The present invention relates to a method for testing the sterility of a plant by checking for the presence and identifying at least one microorganism.

### RELATED APPLICATIONS

This application is a continuation of U.S. application Ser. No. 13/473,016, filed May 16, 2012, which claims priority to Italian patent application M12011A000864, filed May 17, 2011. The content of the foregoing applications is incorporated herein by reference in its entirety.

### FIELD OF THE INVENTION

The present invention relates to a method for testing the sterility of a plant by checking for the presence and identifying at least one microorganism.

More particularly the invention relates to testing and/or monitoring the sterility of a plant, especially a pharmaceutical production or formulation plant, by detecting the presence or absence of microorganisms within all those plant parts which are in contact with the product, without the use of growth medium and without the need to await growing of a microorganism culture.

### BACKGROUND OF THE INVENTION

To date, the procedure for testing the sterility of plants, especially pharmaceutical or nutritional production and formulation plants, is primarily based on the use of methods that entail the analysis of the entire process by means of simulation, known as ‘process simulation’ and a subsequent culturing of the potentially contaminated solutions and a growth based analysis. Examples of such plants are plants that produce pharmaceutical products or products of nutritional value, which require sterile or at least aseptic production units,

According to the PDA Journal of Pharmaceutical Science and Technology, (Technical Report No 28, 2006 Supplement Volume 60, page 14), the term ‘process simulation’ is meant to describe a method of evaluating an aseptic process employing methods which either closely approximate those used for sterile materials using an appropriate material (“without microbiological growth media”), or using a microbiological growth medium (“with microbiological growth media”). The latter is sometimes also referred to as “media fill”. During this simulating process the entire campaign is simulated by repeating all those critical operations which comprise being in contact with the product.

The process simulation comprising the use of microbial growth media, that is also known as media fill, normally includes exposing the microbiological growth medium to product contact surfaces of equipment, container closure systems, critical environments, and process manipulations to closely simulate the same exposure that the product itself will undergo.

Process simulation can be performed just once for the totality of the process (single simulation) or simulations can be performed on the single productive operations (step simulation).

In order to demonstrate that the sterility requirements are met during a production campaign, it is required by the authorities, which certify sterility based on a given protocol, that the number of operations that are performed during one campaign (comprising, for example, production of ten batches) is repeated during process simulation. For example, if testing comprises production of eightbatches, all the steps I should be simulated eight times with the placebo material. Such steps may be, for example, connecting a mixer, taking a sample or filling of the product

Single simulation has the disadvantage that it does not permit identification of the point of contamination in the event of a positive test result indicating contamination (failure of the test), It also requires the use of a single type of material for the entire simulation process, something which is not always possible in that the equipment used for process phases in which the product is in a liquid phase may not be suitable for the use of a powder and vice versa.

Step simulation entails a higher use as analytical process, but this procedure is extremely lengthy and the results of any contamination are not indicated immediately. The entire campaign is simulated by repeating the critical operations in contact with the product.

Following the simulation and, therefore, the passing of the material through the plant, the sterility of the material must be analysed in order to ensure that no microorganisms have been introduced into plant equipment during the production campaign.

To date the sterility check is typically performed using a method that provides for the control of or monitors microbial growth by means of analysis of the turbidity of the growth medium for microorganisms.

When the process simulation is performed with the use of a growth medium, the I growth medium is collected, and sealed containers filled with the medium are then incubated under suitable conditions and temperature. If the growth medium turns turbid, it is concluded that microbial contamination based on growth of a living culture is present.

However, this procedure is extremely lengthy, and the results of contamination are not immediate. Moreoverome organisms will take several days before growing,—with consequent delay in the subsequent production campaign.

When the process simulation is performed with a placebo material, i.e. with a material (other than growth medium) simulating the sterile material used during production, the latter will be collected and growth medium will be added to the placebo or alternatively the placebo may be filtered on a membrane and the membrane is placed inside a bottle with liquid growth medium, which is then incubated and subsequently analysed for the presence or absence of microbial contamination by determining the presence or absence of turbidity in the liquid culture medium.

These procedures, whether they entail the use of placebos or media are very time-consuming. They do not provide immediate results but require time for the growth of the different microorganisms and for checking the turbidity of the growth medium. Because, the positive outcome of the sterility test, i.e. the statement that no microorganism could be identified, must be obtained prior to commencing the subsequent production campaign, this procedure entails a downtime of at least fifteen days for the plant if performed with the usual procedures, linked to the growth in the growth medium.

I media and energy costs add to the delay in production, as well as cost of the entire bottling and growth procedure under the different growth conditions for the different types of microorganism. Furthermore it produces a substantial waste.

In the case of an antibiotics production campaign, there is the further problem that the process simulation cannot be performed immediately at the end of a production campaign, One possible reason is that the presence of residual antibiotic preferably of antibiotics that act on the bacterial replication and growth mechanisms of cells, such as, for example beta-lactam antibiotics, could result in false negative outcomes, linked to a microbial growth inhibition effect caused by the residual antibiotic.

A further disadvantage of the procedures used in the art to date lies in the need to simulate an entire production campaign, repeating every single step as often as was done during the campaign, with consequently extended plant downtime for the production processes.

### SUMMARY OF THE INVENTION

The object of the present invention is therefore to provide a procedure for testing, determining and/or monitoring the sterility of a plant that does not present the abovementioned disadvantages described for the procedures known in the art.

The object indicated above has been achieved by application of a method for testing the sterility of a plant during process simulation, comprising phase a) of detecting the direct presence of at least one vital microorganism characterised in that this phase is performed with the aid of a cytometer, which is able to detect a single cell or a single microorganism.

The problems in the state of the art have been overcome by a new process for determining whether or not the plant is contaminated by one or more living microorganisms. The method comprises the steps of filling the plant production units with placebo material, collecting the placebo material after passage through the plant production units, passing the collected placebo material through a membrane filter suitable to filter any microorganism and detecting the presence or absence of at least one microorganism on said membrane with the use of a cytometer.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and are not intended to be limiting. Other features and advantages of the invention will be apparent from the following detailed description and claims

### DESCRIPTION OF THE DRAWINGS

The characteristics and the advantages of the present invention shall become clear from the detailed description provided below and from the example embodiments provided by way of a non-limiting example, as well as from the accompanying Drawings, wherein:

FIG. 1 is a photograph of a disc-holder, the device used to hold the membrane filter, of Examples 1, 2 and 3.

FIG. 2 is a photograph of the filter system, in miniature, of Example 5.

### DETAILED DESCRIPTION OF THE INVENTION

The invention provides a method for testing the sterility of a plant during process simulation or media fill, comprising a method that includes detecting the direct presence of at least one vital microorganism using a cytometer characterized able to detect single cells, i.e. a single microorganism.

The invention additionally provides a method for determining whether a plant, or more specifically the plant production units used during a production campaign, or a single or several production processes are contaminated by one or more living microorganisms. The absence of living microorganisms in any of the units of the entire production process as result of a test according to the invention with a single cell sensitivity indicates sterility of the production units as well as of the simulated process because sterility is defined as the absence of living microorganisms. According to the invention such a production unit may be any unit that is used in the production process. Such a production process is to be understood as comprising a process of synthesis or formulation or filling or sealing or any mixtures thereof. For example it may comprise steps of weighing, blending, dissolving, crystallising, filtrating, lyophilising, drying, filling and capping. According to the invention the term production unit comprises any unit used in any such process.

Sterility should not only be demonstrated for the end product, but also for the entire production process, even for the entire production campaign. According to the invention this is done by simulating the production processes with a placebo material. The simulation step includes exposing the placebo material to product contact surfaces of equipment, container closure systems, critical environments, and process manipulations to closely simulate the same exposure that the product itself will undergo. To simulate the process the material is preferably filled into a first plant production unit, for example, the storage container of the educts, and subsequently passed through the entire system of plant production units, if several are used. Thus, the placebo material that is used during simulation of the process is exposed to the same environment as the educts, additives and solvents used and the products produced during such a production process or campaign.

According to the invention the placebo material may be of only one type or several different types. The placebo material may be liquid or solid, or it may be a mixture of both according to the requirements of the different process phases and the equipment used therein, just as in a standard process simulation. In any case according to the embodiment of invention, which is directed to determining whether or not a production unit is contaminated, i.e. is not sterile, by determining the presence or absence of at least one microorganism in the placebo material, this placebo material must be sterile. For example, a liquid placebo material may be a sodium chloride solution, and a solid placebo material may be PEG. Any sterile material may be used, unless there are remains of antibiotic substances in the plant production units. If that is the case, care has to be taken not to use a medium containing nutrients, which causes the potentially present microorganism to replicate or grow, as the residual antibiotic could kill those microorganisms that are in replication or growth phase.

The preferred placebo material is water, most preferably WFI, water for injection. WFI is water for the preparation of medicines for enteral administration when water is used as a vehicle and for dissolving or diluting substances or preparations for enteral administration before use. WFI is obtained from water that complies with the regulation on water intended for human consumption, laid down by the competent authority, or from purified water by distillation in a further specified apparatus (see “Note For Guidance On Quality Of Water For Pharmaceutical Use from 2002 under “4. Requirements of the European Pharmacopeia”, 4.2). It contains no more than 0.25 IU of endotoxin per ml. No antimicrobial or other substance has been added. The pH maybe between 4 and 7.5, preferably between 5.0 and 7.0, and most preferably 5.5. The osmolality maybe between 0 and 20.

For example, the WFI which can be purchased from Gibco is characterized by the following parameters:

| Parameter | Limits | Units |
|---|---|---|
| Endotoxin Testing | <= 0.25 | EU/mL |
| Conductivity Measurement | <= 5.0 | us/cm |
| Osmolality | >= 0 to <= 20 | mOsm/Kg |
| Oxidizable Substances | Meets USP requirements | |
| pH | >= 4.0 to <= 7.5 | |
| Sterility Testing | Negative | |

Preferably, the entire placebo material is collected. If the collected placebo material is of solid type, it is dissolved in a suitable, preferably sterile, solution, such as WFI.

The method further comprises a step of analysing the placebo material for presence or absence of a living microorganism by recovering from this placebo material any microorganism present and fluorescent labelling thereof. The solutions containing or consisting of placebo material are preferably passed through a membrane filter. The membrane filter is suitable for filtering a single cell or microorganism from the either liquid placebo material or the solution of dissolved solid placebo material. Preferably, the filter is a 0.3 to 0.5 um, most preferably a 0.4 um pore size membrane.

The dissolving and the filtering step are preferably done in a separate laboratory, suitable for performing the analysing step with the cytometer.

The analysing step preferably comprises washing the membrane filter with a sterile solvent. It is a preferred embodiment wherein the solvent used is water, preferably WFI.

Analysing the solvent either comprises labelling the microorganism with a chromophore in solution, and scanning the solvent with a liquid phase laser scan cytometer (flow cytometry) or comprises filtering the solvent with a filtering device thereby labelling any microorganism recovered on its surface with a chromophore and scanning the filtering device with a solid phase laser scan cytometer (solid phase cytometry) and thereby detecting the presence or absence of at least one microorganism.

If the solvent is filtered with a filtering device suitable for cytometric analysis, the microorganisms may be labelled, preferably with a chromophore, such as a fluorochrome, while being captured on the membrane: The living microorganisms may be labelled according to a procedure based on specific reagents located in the membrane of the filtering device. The microorganisms present in the sample—if any—are retained on the membrane. The membrane is soaked with a specific reagent system which uses enzymatic cleavage of a non-fluorescent substrate to set free a fluorochrome into the cytoplasm of viable cells. Only metabolically active cells, including spores, have the ability to perform this specific cleavage. These cells may be labelled for example with ‘Fluorassure’ reagents.

Subsequently, in a last step the presence or absence of at least one labeled living microorganism is detected with a cytometer, characterized as being able to detect single cells, i.e. a single microorganism, preferably with a laser scan cytometer. Any cytometer which is able to differentiate between no cell and one cell or microorganism is suitable to be used in this method. Currently the preferred option is the use of a solid state laser scan cytometer. Scanning may be obtained by overlapping traces and any cell present on the filter which has been labeled accordingly will be individually and directly detected and counted, thereby determining the presence or absence of living microorganisms in the placebo material, and thereby indicating sterility or contamination of the production units and/or the production process.

It is preferred that the cytometer is a solid phase scan cytometer. It is even more preferred that the 488 nm argon laser scan cytometer is employed.

Because the method is non-destructive, the organism remains viable and could be used for inoculating a new culture if deemed necessary, for any reason.

The new process for determining whether or not the plant production units are contaminated by one or more living microorganisms, or for confirming sterility of the plant production units and the simulated process is further characterized by detecting the presence or absence of a microorganism in a placebo material. The placebo material is used to simulate the process of production preferably without culturing of potentially contaminated material in growth media and awaiting the extended phase of growth and microbial multiplication of single cells potentially present therein.

An advantage of solid phase cytometry is does not require testing for microbial growth in a test culture inoculated with any potentially contaminated test solutions.

In one embodiment the method comprises the additional step of discharging the existing plant production units from the material used during a production campaign, prior to the simulating step. This is the case if the sterility testing method is applied subsequently to a production campaign. Preferably, the step of simulating follows immediately after the step of discharging, because there is no need for an extra step of cleaning the plant and no requirement of removing residual antibiotic substances. This is a great advantage compared to the use of other methods which entail the use of growth media and depend on the microorganisms growth.

The discharging step may be omitted, though, if the sterility test is performed on a new or previously cleaned plant.

In principle any microorganism can be detected if it can be detected by a method sensitive enough to detect a single cell. According to the embodiment wherein the solid phase cytometry is used which is based on the labelling reaction depending on the enzymatic activity of the viable cell, any microorganism may be detected which is still viable when it is recovered at the membrane of the filtering device. According to the invention it is preferred that the microorganism is selected from the group consisting of bacteria, yeast, spores and mould. More preferably the microorganism is selected from the group consisting of Staphylococcus aureus, Pseudomonas aeruginosa, Clostridium sporogenes, Bacillus subtilis, Candida albicans and Aspergillus niger.

It is a preferred embodiment the plant production units are used to produce pharmaceuticals, wherein production is meant to comprise synthesising, formulating, filling, etc. as described above. More preferably, the plant production units are used for the production of antibiotic substances. It is even more preferred that these antibiotic substances are beta-lactam antibiotics.

A beta-lactam antibiotic is defined as a broad class of antibiotics inclusin all antibiotic agents that contain a β-lactam nucleus in their molecular structures. This includes penicillin derivatives (penems), cephalosporins (cephems), monobactams, and carbapenems. Most β-lactam antibiotics work by inhibiting cell wall biosynthesis in the bacterial organism. More specifically, they act by inhibiting the synthesis of the peptidoglycan layer of bacterial cell walls.

β-Lactam antibiotics are analogues of d-alanyl-d-alanine—the terminal amino acid residues on the precursor NAM/NAG-peptide subunits of the nascent peptidoglycan layer. The structural similarity between β-lactam antibiotics and d-alanyl-d-alanine facilitates their binding to the active site of PBPs. The β-lactam nucleus of the molecule irreversibly binds to (acylates) the Ser₄₀₃ residue of the PBP active site. This irreversible inhibition of the PBPs prevents the final crosslinking (transpeptidation) of the nascent peptidoglycan layer, disrupting cell wall synthesis.

In the preferred embodiment the plant is an antibiotic production plant, and the placebo material, as well as the solvent used to dissolve the solid placebo material, does not promote the growing or replicating of the microorganisms, sas growth media would. Preferably, the material used is water, most preferably water for injection (WFI). Because there is no need for an intense cleaning procedure after a production campaign prior to the process simulation in order to remove residuals of antibiotics, in this method, the microorganisms are therefore preferably characterized by surviving the step of simulating the production process with placebo material in presence of residual antibiotic substances.

Wherein the antibiotic substances act by inhibiting the replication phase, such as when beta-lactam antibiotics are used, the microorganisms are characterized by surviving the step of simulating the production process with placebo material in non-replicating phase in the presence of such residual antibiotic substances such as, for example, beta-lactam, and the placebo material is material which does not promote growth or replication of the microorganisms.

This was demonstrated, by an inoculation of a known amount of microorganisms (see examples) in antibiotic and antibiotic mixed with placebo, respectively solid or liquid, and testing the recovery for microorganisms at successive time intervals up to the time required to complete the execution of the process simulation. The applied method did not kill the microorganisms.

In the most preferred embodiment, the method for determining whether at least one plant production unit used during a production campaign, or single or several production processes that produced a beta-lactam antibiotic is contaminated by at least one living microorganism or not, is performed by determining the presence or absence of at least one living microorganism The method preferably comprises the steps of (a) discharging the plant production units from substances required during the production campaign; (b) filling the production units with placebo material, wherein the placebo material is sterile and does not promote the replication of microorganisms; (c) passing the placebo material through the entire number of production units required for a production process or campaign; (d) collecting the placebo material, (e) passing the placebo material through a membrane filter, thereby recovering from the placebo material any microorganism present; (f) washing the filter with a solvent, (g) passing the solvent through a filter device thereby recovering and labelling any living microorganism therein; (h) detecting the labelled living microorganisms with a laser scanning cytometer, preferably a solid phase laser scanning cytometer; thereby determining the presence or absence of at least one living microorganisms in the production units. In some embodiments, the method neither comprises a cleaning step for removing residual antibiotics in between the discharging step (a) and the filling step (b); nor comprises a step of testing for microbial growth in a test culture inoculated with any potentially contaminated solution.

In a different embodiment according to the invention the placebo material may not be sterile. The placebo material must be sterile if sterility of the production unit, i.e. the presence or absence of a single microorganism has to be determined. If, however, the method is used to detect certain types of microbiological organisms, such as pathogens, and the plant is not required to be sterile per se, the placebo material need not be sterile either. In such a case either the amount of microorganisms or the kind of microorganisms may be of interest. If presence of microorganisms has been determined by the method, they can also be quantified. If the identity of these is of interest, additional steps will have to be performed to identify the type of microorganism. Given that the microorganism is still alive at the time of detection it is one option to start a microculture in a lab. Iternatively the microorganism might be identifiable under a microscope, or via DNA analysis. Such a method might be suitable for a food producing or packaging plant.

For the purposes of the present invention, “detecting the direct presence of at least one v microorganism” means to confirm sterility without envisaging the growth and microbial multiplication phase.

As will indeed be apparent from the Examples provided hereunder, this method has proven to be surprisingly effective in ascertaining the presence of a diversity of microorganisms such as bacteria, yeast, moulds and spores in pharmaceutical production plants and in food production plants, without requiring the subsequent growth and microbial multiplication phase.

According to a preferred embodiment, the method for testing the sterility of a plant during process simulation comprises the phase a) of detecting the presence of at least one vital microorganism with the aid of a laser scanning cytometer, without envisaging the growth and microbial multiplication phase.

According to a preferred embodiment, the method for testing the sterility of a plant during process simulation comprises the phase a) of detecting the direct presence of at least one vital microorganism with the aid of a laser scanning cytometer.

The method according to the present invention allows afoar all the microorganisms to be detected in the space of a few hours (around 3-4 hours) and of permitting an individual microorganism that may be present to be detected and subsequently identified.

A further advantage of this method according to the invention is the fact that it is non-destructive. In the rare case that a microorganism has been detected, it will still be alive and it is possible to determine its identity, either directly under a microscope or by starting a culture now, in a case where this seems to be required by protocol or by law or because optical identification is not possible.

In addition, the greater sensitivity of the method permits, in a single analysis, the detection of not only those microorganisms able to grow in standard growth mediums, but also microorganisms such as yeast, moulds and spores that do not grow under identical temperatures and conditions, with consequent cost and time reductions.

Surprisingly, in a preferred embodiment, the method according to the present invention further comprises a phase b) for the collection of the at least one microorganism, to be carried out before phase a).

In an even more preferred embodiment, phase b), according to the method of the present invention, comprises recovering at least one microorganism on a membrane filter, washing the membrane filter with a solvent and filtering the solvent with a filtering device.

Washing of the membrane filter can be conveniently carried out with reduced volumes of solvent (e.g. water). The subsequent filtration of the solvent through a filtering device permits analysis with a cytometer, for example with a solid-phase cytometer.

This recovery procedure would in any case also permit the use of a liquid-phase cytometer.

The cytometer can therefore be a laser scanning cytometer, a solid-phase cytometer or a liquid-phase cytometer. Preferably, the cytometer of the method according to the present invention is a laser scanning cytometer.

Even more preferably, the cytometer has a 488 nM argon laser and performs a measurement of fluorescence.

In addition, by applying this method, process simulation or media fill can be performed at the end of the production campaign and with no need to proceed with the cleaning of the plant, thus reducing the costs and speeding up the procedure. Indeed, microbial growth is not envisaged for this method and there can therefore be no inhibition of the beta-lactam antibiotics (or antibiotics that are only active during the replication phase). Performing process simulation immediately following an actual production campaign guarantees a closer resemblance of reality, as a potential contamination otherwise could have been deleted during such a cleaning process. The method according to the invention allows for a more reliable testing of the actual production and therefore improves the guarantee of sterility of the processes.

According to the method of the invention, wherein the method is performed on an antibiotic production plant, immediately after an antibiotic production campaign, those microorganisms should be used, which in non-replicating phase survive in the presence of the residual antibiotic during the execution of process simulation with placebo, so as to reach the cytometer reading alive.

In one embodiment of the invention it is preferred that the microorganism is labelled with a chromophore.

Under another aspect, the present invention relates to a method for testing the sterility of a plant during process simulation, which comprises the phase of detecting the presence of at least one vital microorganism that is labelled with a chromophore.

According to yet another preferred embodiment, the method according to the present invention permits detection of the direct presence of at least one vital microorganism. This microorganism is preferably selected from the group consisting of bacteria, yeast, spores and moulds.

Advantageously, the method according to the present invention permits detection of the direct presence of microorganisms selected from the group consisting of Staphylococcus aureus, Pseudomonas aeruginosa, Clostridium sporogenes, Bacillus subtilis, Candida albicans and Aspergillus niger.

According to a preferred embodiment, the method for testing the sterility of a plant according to the present invention is a pharmaceutical production plant.

According to another preferred embodiment, the method for testing the sterility of a plant according to the present invention is an antibiotics production plant.

According to yet another preferred embodiment, the method for testing the sterility of a plant according to the present invention is a beta-lactam antibiotics production plant.

It is a preferred embodiment of any of the methods according to the invention wherein the plant is a pharmaceutical or nutritional production plant.

It is even more preferred that the plant is an antibiotics production plant. And it is especially preferred that such plant is a beta-lactam antibiotics production plant.

The following are embodiments meant to describe different aspects of the invention.

1. Method for testing the sterility of a plant during process simulation or media fill, comprising phase a) of detecting the direct presence of at least one vital microorganism characterised in that phase a) is performed by means of a cytometer.
2. The method according to embodiment 1, wherein said cytometer is a laser scanning cytometer.
3. The method according to embodiment 1, wherein said at least one vital microorganism is labelled with a fluorescent probe.
4. The method according to any one of embodiments 1-3, wherein said method further comprises phase b) of sampling of said at least one microorganism, to be performed before phase a).
5. The method according to embodiment 4, in which phase b) comprises recovering said at least one microorganism on a membrane filter, washing said membrane filter with a solvent and filtering said solvent with a filtering device.
6. The method according to any one of embodiments 1-5, wherein said at least one microorganism is selected from the group comprising bacteria, yeast, spores and mould.
7. The method according to any one of embodiments 1-6, wherein the laser in said cytometer is a 488 nM argon laser.
8. The method according to any one of embodiments 1-7, wherein said plant is a pharmaceutical production plant.
9. The method according to any one of embodiments 1-7, wherein said plant is an antibiotics production plant.
10. The method according to embodiment 9, wherein said plant is a beta-lactam antibiotics production plant.

Example embodiments of the present invention are provided by way of illustration below.

### EXAMPLES

### Example 1

### Analysis of the Sterility of the Production Process: Liquid Part

Plant sterility is checked by carrying out a simulation of the entire production process (process simulation). This production process provides for both a phase wherein the reagents are in the liquid state (liquid part) and a phase wherein the reagents are in the solid state (solid part).

This “liquid part” involves the first part of the production process: the synthesis, and is described in the present Example.

Equipment such as sterilising filters, reactors, crystallisers, filter-dryers is used in the liquid part of the process. The liquid part terminates with the formation of the powder in the filter-dryer.

A placebo is used to execute both the liquid part and the solid part, which in the case of the liquid part is a physiological solution.

The placebo is transferred into the plant and the synthesis phase of the production process, which comprises the following sterilising filtration, crystallisation, drying, exhaust phases, is simulated.

After passing through the plant, the placebo is collected and filtered for analysis of the microorganisms.

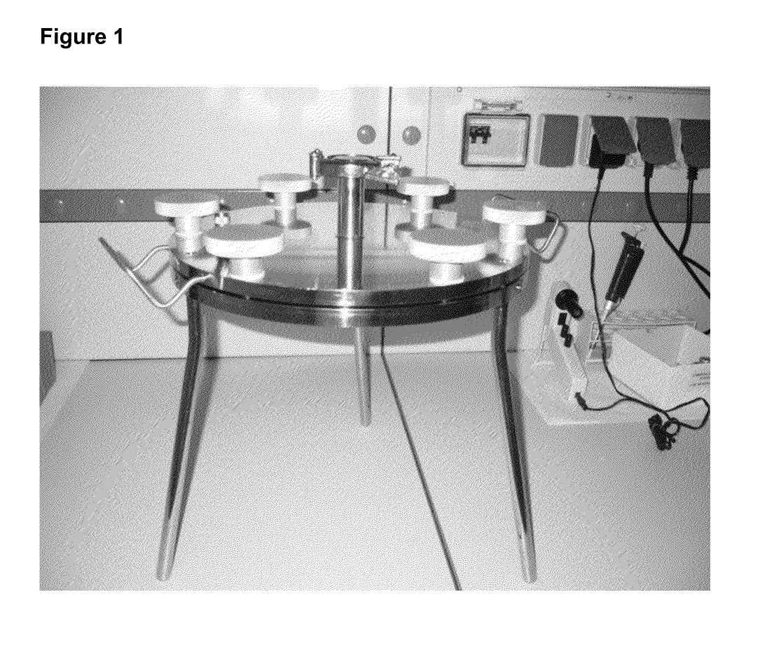

Analysis of the entire placebo is performed by means of filtration using a disc holder, a device that consists of two stainless steel discs fixed together by screws (FIG. 1) and a retention membrane for the microorganisms. This membrane is a 30 cm diameter nylon Utipor N⁶⁶ membrane with pore size of 0.45 μm.

For the liquid part, the physiological solution that is contained in the filter-dryer is filtered.

### Example 2

### Analysis of the Sterility of the Production Process: Solid Part

The second part of the production process or “solid part” is described in the present Example, and involves the product grinding, mixing and bottling phases. Equipment in which the product is in powder phase and which are not suitable for the processing of liquids are therefore used.

The solid part terminates with the bottled product.

In executing the solid phase, the placebo is PEG 8000 in sterile powder and is transferred into the plant in which the abovementioned production process phases are simulated.

After passing through the plant, the placebo is collected, poured into the mixer, dissolved with sterile WFI (water for injection or milli-q sterile water) and then filtered for analysis of the microorganisms.

Analysis of the placebo is performed by filtration using a disc holder (FIG. 11) and retention membrane for the microorganisms as per the liquid part.

### Example 3

### Treatment of the Retention Membrane for Microorganisms and Laser Scanning Cytometer Analysis

The retention membrane for microorganisms is removed and treated to check the sterility of both the “liquid part” and the “solid part” of the production process once filtration using a disc holder as described in Examples 1 and 2 has been performed.

The disc holder containing the membrane is place in the sterile chamber and subsequently under laminar flow (class A)

The disc holder is positioned horizontally with the supports downwards and the fastening screws upwards

The disc holder is opened by rotating the screws and the upper disc is lifted, leaving the membrane resting on the lower disc. The membrane is cut into four quadrants using a sterile blade, or sterile scissors.

A bottle containing 900 ml of sterile WFI is opened and sterile forceps are used to remove a quadrant that is placed inside the bottle that is closed.

The procedure is repeated for the other three quadrants in as many separate bottles.

A negative control is performed at the same time as the analysis by opening a bottle of TSB medium and simulating the membrane insertion operations.

The bottles containing the WFI-immersed membrane are hand-shaken for two minutes and subsequently sonicated for two minutes.

The WFI contained in the bottle is filtered and analysed as per the standard laser scanning cytometer protocol for the sterility test.

More than one filtering is performed using one filtering device for no more than 250 ml of solution. In this way, the risk of particle overload is reduced. On the other hand, it is possible to filter a lesser quantity in the case of slow filtrations and thus use multiple filtering devices.

In so doing, the 900 ml of physiological solution is filtered:
250 on a first filtering device
250 on a second filtering device
250 on a third filtering device
150 on a fourth filtering device
   100 ml of WFI are added to the empty bottle still containing the membrane and hand-shaken for one minute.

These last 100 ml are filtered in the fourth filtering device.

Steridilutors (Millipore) are used to add the WFI and the filtrations to the filtering devices. In this way, opening of the bottle, having a septum cap, is avoided.

The same procedure is followed for the other three bottles.

All the filtering devices thus obtained from the standard laser scanning cytometer protocol for the sterility test are analysed.

The laser scanning cytometer is capable of determining and counting a very low number of microorganisms, in the order of individual cells with extremely high detection sensitivity. The instrument works on Solid-Phase Cytometry by fluorescence. This technology is capable of discriminating between living and dead microorganisms and the auto-reflecting particles.

The samples are collected by filtration on a membrane and analysed with the scanning cytometer.

A negative control is also carried out by filtering 100 ml of WFI.

The following procedure is subsequently performed to confirm the sterility test.

The empty bottles, containing the quadrants of the membrane, are filled with growth medium to also collect any residual microorganisms on the membrane or wall of the bottle.

The growth mediums are as follows:

Fluid Thioglycollate Medium (TIO):

| L-Cystine | 0.5 | g |
|---|---|---|
| Agar | 0.75 | g |
| Sodium chloride | 2.5 | g |
| Anhydrous glucose monohydrate | 5.5 g/5.0 g | |
| Soluble yeast extract | 5.0 | g |
| Casein peptone | 15.0 | g |
| Sodium thioglycollate | 0.5 g (or 0.3 ml thioglycolic acid) | |
| Resazurin solution (1 g/l) | 1 | ml |
| Purified water | 1 | litre |

| | |
|---|---|
| pH after sterilisation | 7.1 ± 0.2 in 100 ml bottles |
| | with septum cap. |

### Soy-Bean Casein Digest Medium (TSB):

| Casein peptone | 17.0 | g |
|---|---|---|
| Soy peptone | 3.0 | g |
| Sodium chloride | 5 | g |
| Dipotassium hydrogen phosphate | 2.5 | g |
| Anhydrous glucose monohydrate | 2.5 g/2.3 g | |
| Purified water | 1 | litre |

| | |
|---|---|
| pH after sterilisation | 7.3 ± 0.2 in 100 ml bottles |
| | with septum cap. |

The following procedure is followed with the aid of a steridilutor:
900 ml of TSB plus 50 ml of Penase (Sigma Penicillinase cat. No. P0389 from Bacillus Cereus Type) are added to two bottles;
900 ml of TIO plus 50 ml of Penase are added to the other two bottles

The bottle caps, now pierced, are replaced with unpierced, sterile caps.

The TSB medium is incubated at 20-25° C. for 14 days and the TIO medium at 30-35° C. for 14 days.

### Example 4

### Validation Procedure

The method described in Example 3, readjusted on a reduced scale in the laboratory, is applied for the convalidation.

Titrated microbe strains will also be added at the initial filtering phase to check the final recovery on completion of all the analytical phases.

The microbe strains tested (lyophilised and titrated <100 ufc) are summarised in Table 1:

| | | Incubation conditions | | |
|---|---|---|---|---|
| Microorganisms | | Growth | Temper- | Maximum |
| Species | Strain | medium | ature | duration |
| Staphylococcus aureus | ATCC 6538 | TIO/TSA | 30-35° C. | 3 days |
| Pseudomonas | ATCC 9027 | TIO/TSA | (aerobic) | |
| aeruginosa | | | | |
| Clostridium | ATCC 19404 | TIO/TSA | 30-35° C. | 3 days |
| sporogenes | | | (anaerobic) | |
| Bacillus subtilis | ATCC 6633 | TSB/TSA | 20-25° C. | 5 days |
| Candida albicans | ATCC 10231 | TSB/SGA | | |
| Aspergillus niger | ATCC 16404 | TSB/SGA | | |

| | | |
|---|---|---|
| Staphylococcus | From section | as per Staphylococcus |
| epidermidis | controls | aureus |
| Ochrobactrum | From section | as per Staphylococcus |
| anthropi | controls | aureus |

### Filtration of the Placebo Through the Membrane

The quantity of placebo (PEG) subjected to filtration and analysis is 5 kg, which is dissolved in 200 ml of WFI.

The membrane used is in 29.3 cm diameter Ultipor 66 nylon with pore size of 0.45 μm.

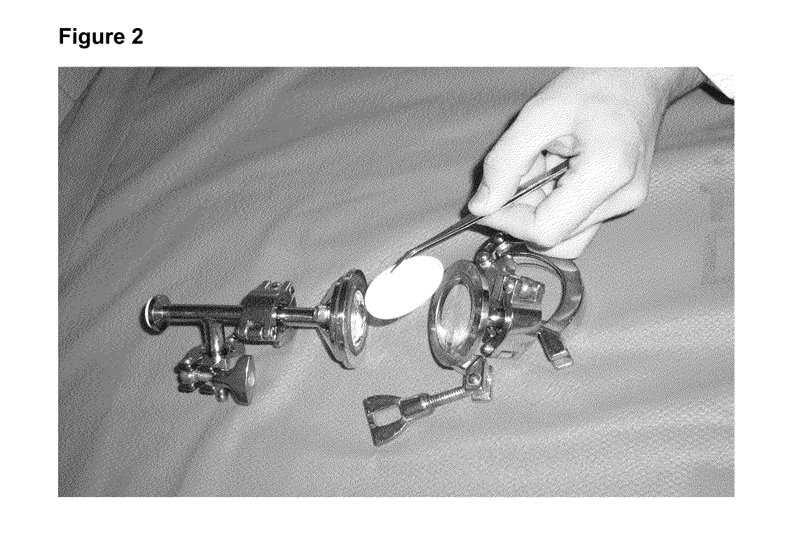

The convalidation is performed on a miniature filter system (FIG. 2), by proportioning all the reagents as described in Table 2.

TABLE 2

| | | | Area to | | |
|---|---|---|---|---|---|
| | | | undergo | | |
| | Membrane | Membrane | conval- | Kg of | Dissolution |
| | diameter | area | idation | placebo | volume |
| Plant: | 29.3 cm | 674 cm² | 168.5* cm² | 5 | 200 litres |
| Conval- | 4.7 cm | 17 cm² | 17 cm² | 0.5** | 5 litres |
| idation | | | | | |

*The membrane is cut into four quadrants immersed in four separate bottles of medium. It is therefore ¼ of the total area
**The plant/convalidation proportion factor is 10, in fact 168.5: 17 = 9.9 0.5 kg of placebo, dissolved in 5 litres of sterile WFI at T < 30°, is then filtered using a smaller filter system with a 4.7 cm diameter membrane in Utipor 66 nylon and pore size of 0.45 μm.

On the basis of production experience, around 2.5 kg of product are left in the plant. 5 kg of powder, comprising approximately 2.5 kg of PEG and 2.5 kg of antibiotic will therefore be analysed in the plant. In this specific case, the chosen antibiotic is cefuroxime.

In the laboratory, the following procedure is followed:
100 g of Cefuroxime are dissolved in 1 litre of sterile WFI at T<30° C. This is repeated five times to achieve the dissolution of 0.5 kg of cefuroxime.
The solution is filtered in a laminar flow cabinet using vacuum pump and sterile filtration system (FIG. 2)
The membrane is washed with 100 ml of sterile WFI, in which <100 ufc of the microbe strain to be tested have been inoculated
In parallel, the inoculum is plated on three separate TSA/SGA plates, depending on the type of strain, as a control for the inoculated ufcs.

### Solid Medium: TSA:

| | |
|---|---|
| Casein peptone | 15 g/l |
| Soy flour peptone | 5 g/l |
| Sodium chloride | 5 g/l |
| Agar | 15 g/l |

Solid Medium: SGA with Chloramphenicol:

| | |
|---|---|
| Casein peptone | 10 g/l |
| Glucose | 40 g/l |
| Agar | 15 g/l |

Recovery of the Microorganisms from the Membrane

The membrane, treated as indicated above, is placed in 100 ml of sterile WFI.

It is hand-shaken for two minutes and then sonication in an ultrasonic bath is performed (50 kHz frequency) for a further two minutes.

Negative control: in parallel, a sterile membrane is immersed in 100 ml of WFI, from the same batch used for the membrane of the test, and is treated in the same way.

Analysis with Laser Scanning Cytometer

Filtering of the 100 ml of WFI, in which the microorganisms on the filtering supports for the laser scanning cytometer have been recovered, then takes place. To prevent the accumulation of autofluorescent particles on the membrane, at least four filtering devices are used, in which the WFI is subdivided. The bottle still containing the membrane from which the microorganisms were recovered is washed with a further 20 ml of WFI, shaking for one minute. The 20 ml are filtered on a filter support.

Also performed are:
a negative control intended as a blank control of the reagents used including the WFI in which the sterile membrane has been inserted
a positive control of the original inoculum to check the ufcs/inoculated

The laser scanning cytometer analysis is performed as per the standard method

On termination of the reading, it is checked that:
the blank control does not present microbial forms; and
the sum of the microorganisms counted in the filtering devices obtained from the recovered water of the original membrane is ≧70% of those counted in the control inoculum.

### Further Analysis of the Original Membrane

At this point, the empty bottle containing the membrane is filled with 90 ml of the TIO/TSB growth medium, depending on the type of strain to be tested, to also recover any residual microorganisms on the membrane or wall of the bottle. 5 ml of Sigma penase 100 UI/ml were preventively added to the mediums.

The medium is incubated at optimal growth temperature and for the time envisaged for the specific strain being tested.

Negative control: in parallel, 90 ml or medium are added to the bottle containing the membrane used for the negative control.

At the end of the incubation period, any microbial growth in the bottle containing the test membrane is detected by medium turbidity analysis.

The broth cultures are plate-seeded in such a way as to have readable colonies and the purity and the type of strain are verified by means of microbial identification.

Recovery of the Microorganism after Laser Scanning Cytometer Reading

At the end of the laser scanning cytometer reading, the membranes of each filtering device are left to grow in 90 ml of TIO/TSB medium, depending on the strain being tested, to which 5 ml of penase 100 UI/ml has been added.

The medium is incubated at optimal growth temperature and for the time envisaged for the specific strain being tested. At the end of the incubation period, microbial growth is detected by medium turbidity analysis.

The broth cultures are plate-seeded in such a way as to have readable colonies and the purity and the type of strain are verified by means of microbial identification.

The morphology of the colony must be unique and typical, and the same applies to the freshly slide analysed microorganisms.

### Interpretation of the Results

The rapid method of process simulation analysis is deemed convalidated if:
the number of microorganisms recovered from the membrane, read with the laser scanning cytometer is ≧70% of the reading of the original inoculum;
the number of microorganisms recovered from the membrane, read with the laser scanning cytometer is ≧70% of the original plate-seeded inoculum;
the negative controls present no anomalies;
the growth of the residual microorganisms in the original membrane is manifested in the required timeframes;
the recovery of the microorganisms from the laser scanning cytometer membrane is consistent; and
the identification of the microorganisms does not present anomalies.

In particular, the results relating to the microbial strains are recorded in Table 3:

| | Number of | Recovery | | Recovery | |
|---|---|---|---|---|---|
| | micro- | compared to | | compared to | |
| | organisms | the positive | | the positive | |
| | recorded | plate control | | cytometer control | |
| Strain | (TEST) | no. in CPP | % | no. in CPC | % |
| Staphylococcus | 66 | 67 | 98 | 69 | 96 |
| aureus | | | | | |
| Pseudomonas | 24 | 26 | 92 | 26 | 92 |
| aeruginosa | | | | | |
| Clostridium | 18 | 21 | 86 | 20 | 90 |
| sporogenes | | | | | |
| Bacillus | 85 | 41 | 207 | 91 | 93 |
| Subtilis | | | | | |
| Candida | 63 | 65 | 97 | 67 | 94 |
| albicans | | | | | |
| Aspergillus | 49 | 50 | 98 | 52 | 94 |
| niger | | | | | |
| Staphylococcus | 55 | 56 | 98 | 58 | 95 |
| epidermidis | | | | | |
| Ochrobactrum | 38 | 39 | 97 | 41 | 93 |
| anthropi | | | | | |

The results recorded show that the analysis method is suitable as a process simulation test sterility method for all types of production sections: solids, liquids and crystals sections.

The advantages achieved by means of the procedure of the present invention are clear from the detailed description and from the above examples. In particular, said procedure has proven to be surprisingly and advantageously suitable for use in pharmaceutical production plants. At the same time, this method, being rapid and extremely easy to perform, can also be advantageously used in other types of plants in which sterility is essential, for example food industry plants.

Additional embodiments are within the claims.

1. A method for determining whether at least one plant production unit used during a production process is contaminated by at least one living microorganism or not, comprising
a. simulating the production process with placebo material, wherein the placebo material is sterile and
b. analysing the placebo material used during said step of process simulation for presence or absence of a living microorganism by recovering from said placebo material any microorganism present and labelling any living microorganism thereof, and detecting the labelled living microorganisms with a cytometer,
thereby determining the presence or absence of living microorganisms in the production units.

2. The method according to claim 1,
characterized in that the method does not comprise a testing for microbial growth in a test culture inoculated with any potentially contaminated test solutions.

3. The method according claim 1, wherein the step of simulating the production process comprises
a. filling the plant production units with placebo material,
b. passing the placebo material through the plant production units and
c. collecting the placebo material used.

4. A method according to claim 1 comprising the additional first step of discharging the plant production units from the substances required during a production campaign, prior to the step of simulating the production process with placebo material.

5. A method according to claim 1 wherein the microorganism is selected from the group consisting of bacteria, yeast, spores and mould.

6. A method according to claim 1 wherein the microorganism is selected from the group consisting of Staphylococcus aureus, Pseudomonas aeruginosa, Clostridium sporogenes, Bacillus subtilis, Candida albicans, and Aspergillus niger.

7. The method according to claim 1 wherein analysing the placebo material comprises
a. passing the placebo material through a membrane filter, and thereby recovering any microorganism present
b. washing said membrane filter with a solvent and
c. analysing said solvent either
by labelling the microorganism with a chromophore in solution, and scanning the solvent with a liquid phase laser scan cytometer or by filtering said solvent with a filtering device, labelling any microorganism recovered on its surface with a chromophore and scanning the filtering device with a solid phase laser scan cytometer and thereby detecting the presence or absence of at least one microorganism.

8. A method according to claim 7 wherein the solvent is water.

9. A method according to claim 1 wherein the cytometer is a laser scan cytometer.

10. A method according to claim 1 wherein the cytometer is a 488 nm argon laser scan cytometer.

11. A method according to claim 1 wherein the plant production units are used to produce or formulate pharmaceuticals.

12. A method according to claim 1 wherein the plant production units are used to produce or formulate antibiotics and the microorganisms are characterized by surviving the step of simulating the production process with placebo material in presence of residual antibiotic product produced in said production units, and
the placebo material is material which does not promote growth or replication of said microorganisms

13. A method according to claim 1
wherein the plant production units are used to produce or formulate beta-lactam antibiotics
and the microorganisms are characterized by surviving the step of simulating the production process with placebo material in non-replicating phase in presence of residual beta-lactam antibiotic product produced in said production units, and
the placebo material is material which does not promote growth or replication of said microorganisms.

14. A method for determining whether at least one plant production unit used during a production campaign which produced a beta-lactam antibiotic is contaminated by at least one living microorganism or not, by determining the presence or absence of at least one living microorganism, comprising
a. discharging the plant production units from substances required during the production campaign
b. filling the production units with placebo material, wherein the placebo material is sterile and does not promote the replication of microorganisms,
c. passing the placebo material through the entire number of production units required for a production process or campaign,
d. collecting the placebo material,
e. passing the placebo material through a membrane filter, thereby recovering from said placebo material any microorganism present,
f. washing the filter with a solvent
g. passing the solvent through a filter device thereby recovering any living microorganism therein and labelling any living microorganism therein
h. detecting the labelled living microorganisms with a laser scanning cytometer
thereby determining the presence or absence of at least one living microorganism in the production units,
characterized in that the method neither comprises a step of removing residual antibiotics in between the discharging step a) and the filling step b); nor comprises a step of testing for microbial growth in a test culture inoculated with any potentially contaminated solution.

15. The method according to claim 1 wherein the step of analysing the placebo material comprises dissolving any solid placebo material before recovering said at least one microorganism.

16. A method according to claim 1 wherein the placebo material is water for injection (WFI).

17. The use of a solid phase laser scan cytometer for the testing of sterility of a production process.

18. The use of a solid phase laser scan cytometer for the testing of sterility of an antibiotic production process.
